(19) United States
(12) Patent Application Publication
(10) Pub. No.: US 2016/0326547 A1
(43) Pub. Date: Nov. 10, 2016

(54) TRANSGENIC PRODUCTION OF HEPARIN

(71) Applicant: LFB USA, INC., Framingham, MA (US)

(72) Inventors: Harry M. Meade, Newton, MA (US); William G. Gavin, Dudley, MA (US)

(73) Assignee: LFB USA, INC., Framingham, MA (US)

(21) Appl. No.: 15/108,061

(22) PCT Filed: Dec. 19, 2014
(86) PCT No.: PCT/US14/71471

§ 371 Date: Jun. 24, 2016

Related U.S. Application Data
(60) Provisional application No. 61/920,505, filed on Dec. 24, 2013.

(51) Int. Cl. C12N 15/85; C12P 19/26; A01K 67/027; A61K 31/727

(52) U.S. Cl. CPC C12N 15/8509; A61K 31/727; C12P 19/26; A01K 67/0275; C12N 2015/8518; A01K 2227/101; A01K 2227/102; A01K 2267/02; A01K 2217/15

(57) ABSTRACT

The disclosure provides methods, cells and transgenic non human mammals for the production of heparin. Specifically, the method comprising providing a transgenic non human mammal or mammary epithelial cells modified to express one or more heparin biosynthesis enzymes, and harvesting heparin produced. Further provided are the cells and transgenic non human mammals thereof as well as the heparin obtained from these cells or transgenic non human mammals.

### RELATED APPLICATIONS

This application claims the benefit under 35 U.S.C. §119(e) of U.S. Provisional Application Ser. No. 61/920,505, entitled “Transgenic Production of Heparin,” filed on Dec. 24, 2013, which is incorporated by reference herein in its entirety.

### FIELD OF THE INVENTION

The disclosure relates to the field of transgenic production of biological compounds.

### BACKGROUND OF THE INVENTION

Heparin is a complex glycosaminoglycan that is widely used as an anticoagulant. Heparin is generally harvested from animal intestines or bovine lungs. However, there have been issues with contamination of heparin. New methods for the production of heparin are needed therefore.

### SUMMARY OF THE INVENTION

In one aspect, the disclosure provides methods, cells and transgenic mammals for the production of heparin. In one embodiment, transgenic non human mammals that produce heparin in their milk are provided. In one embodiment, a method of producing heparin, the method comprising providing a transgenic non human mammal that has been modified to express one or more heparin biosynthesis enzymes in its mammary gland, and harvesting heparin from the milk produced by the mammary gland of the transgenic mammal is provided. In one embodiment, a method of producing heparin, the method comprising providing a transgenic non human mammal that has been modified to express one or more heparin biosynthesis enzymes and a core protein in its mammary gland, and harvesting heparin from milk produced by the mammary gland of the transgenic mammal is provided.

In another aspect, a method of producing heparin, the method comprising providing mammary epithelial cells that have been modified to express one or more heparin biosynthesis enzymes, and harvesting heparin from the mammary epithelial cells is provided. In one embodiment, a method of producing heparin, the method comprising providing mammary epithelial cells that have been modified to express one or more heparin biosynthesis enzymes and a core protein, and harvesting heparin from the mammary epithelial cells is provided.

In another aspect, a transgenic non human mammal that has been modified to express one or more heparin biosynthesis enzymes in its mammary gland is provided. In one embodiment, a transgenic non human mammal that has been modified to express one or more heparin biosynthesis enzymes and a core protein in its mammary gland is provided.

In another aspect, mammary epithelial cells that have been modified to express one or more heparin biosynthesis enzymes are provided. In one embodiment, mammary epithelial cells that have been modified to express one or more heparin biosynthesis enzymes and a core protein are provided.

In one embodiment, the transgenic mammal is an ungulate. In another embodiment, the ungulate is a goat. In another embodiment, the ungulate is a bovine.

In an embodiment of any of the methods, cells or mammals provided, the heparin biosynthesis enzyme is selected from the group consisting of tetrasaccharide producers, repeating unit producers, repeating unit modifiers, epimerizers, sulfation enzymes and supporting enzymes. In another embodiment, the tetrasaccharide producer is XTII, GalT-1, or GlcAT-1. In another embodiment, the repeating unit producers are EXT1 polymerase and EXT2 polymerase. In another embodiment, the repeating unit modifiers are NDSTI and NDSTII. In another embodiment, the epimerizer is C5 epimerase. In another embodiment, the sulfation enzyme is 3-OST, 6-OST or 2-OST. In another embodiment, the supporting enzyme is UDPGDH.

In another embodiment of any of the methods, cells or mammals provided, one or more of the heparin biosynthesis enzymes as well as the core protein have been modified such that a GPI (GlycosylPhosphatidylInositol) anchor has been manipulated. In one embodiment, a GPI anchor is added to the core protein to target the membrane. In another embodiment, a GPI anchor is deleted to allow secretion into the milk. In another embodiment, the heparin biosynthesis enzymes and core protein have been modified such that the heparin and biosynthesis enzymes are produced in fat globule membranes. In another embodiment, one or more of the heparin biosynthesis enzymes are under the control of a milk promoter. In another embodiment, the milk promoter is a goat beta casein promoter.

In one aspect, heparin produced according to any of the methods provided is provided.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the FIGURE. The invention is capable of other embodiments and of being practiced or of being carried out in various ways. Also, the phraseology and terminology used herein is for the purpose of description and should not be regarded as limiting.

### BRIEF DESCRIPTION OF THE DRAWINGS

The FIGURE is illustrative only and is not required for enablement of the disclosure.

FIG. 1 provides a schematic representation of the Heparin biosynthesis pathway.

### DETAILED DESCRIPTION OF THE INVENTION

In one aspect, the disclosure provides methods, cells and transgenic non human mammals for the production of heparin. While it has previously been reported that administration of heparin can cause side effects, which can be sometimes very serious, such as bleeding, petechiae, purpura, or ecchymosis, it is surprisingly demonstrated herein that low and medium doses of systemic and mammary infused heparin have minimal effects on health and milk production in mammals, allowing heparin production in transgenic mammals.

Heparin is a member of the glycosaminoglycan (GAG) family which consists of polyanionic, polydisperse, linear polysaccharides made up of repeated disaccharide units. The main disaccharides in heparin are L-iduronic acid (IdoA) and N-acetyl-D-glucosamine (GlcNAc), or D-glucuronic acid (GlcA) and N-acetyl-D-glucosamine (GlcNAc). However, there is significant heterogeneity in the heparin polymer chain, due to the further modification of these disaccharides (see description of biosynthesis below). Heparin typically consists of about 10-200 disaccharides with a weight, generally between 3 kDa to 40 kDa (Sasisekharan et al. Curr Opin Chem Biol 4:626-631 (2000)). GAGs such as heparin can be covalently attached to serine residues of a core protein, used as the anchor onto which the sugar synthesis takes place, resulting in a glycoconjugate referred to as a proteoglycan.

Heparin is generally found on the outer membrane of cells and in the extracellular matrix surrounding cells. Heparin interacts with a number of heparin-binding molecules to regulate many biological processes including, e.g., cell growth, cell differentiation, immunity, metabolism, cell signaling, inflammation, blood coagulation, and cancer. Heparin and heparin-like molecules have been identified in both invertebrates and vertebrates, and the molecules isolated from many species seem capable of eliciting at least some of the same biological processes, e.g. anti-coagulation (see, e.g., Medeiros et al. Biochim Biophys Acta 1475(3): 287-294 (2000) and Pejler et al. J Biol Chem 262(24): 11413-21 (1987)).

The involvement of heparin in important biological processes, especially blood coagulation, inflammation, and cancer, has led to the use of heparin as a drug. The role of heparin in coagulation has been extensively studied. Heparin is a naturally occurring anticoagulant produced by basophils and mast cells. Heparin acts as an anti-coagulant by preventing the formation of clots and extension of existing clots within the vascular system. This is accomplished through binding of heparin to antithrombin III (AT), which causes a conformational change which results in AT activation. Active AT then inactivates thrombin and other proteases involved in blood clotting, blocking initial clotting or further clotting. Heparin is currently widely used as an anti-coagulation drug, with over 100 metric tons being used annually (see e.g. Laremore et al. Curr Opin Chem Biol 13(5-6): 633-640 (2009)). Heparin is often indicated, e.g., for deep-vein thrombosis and pulmonary embolism, acute coronary syndrome, atrial fibrillation, cardiopulmonary bypass, hemofiltration, and for indwelling central or peripheral venous catheters. As heparin interacts with many heparin-binding proteins that regulate other processes besides coagulation, clinical trials involving heparin treatment are in progress for many other diseases, e.g., adult respiratory distress syndrome, allergic rhinitis, asthma, and inflammatory bowel disease. Additionally, heparin has also been shown to have anti-metastatic properties in animal models, making it potentially useful for cancer treatment as well (Borsig et al. Proc Natl Acad Sci USA 98(6): 3352-3357 (2000)).

Generally, heparin for use in patients is prepared by isolating heparin naturally occurring in slaughter animals, as human heparin is structurally and functionally similar to other mammalian heparins (see, e.g., Linhardt et al. Biochemistry 31(49): 12441-5 (1992)). However, in 2008 contamination of the heparin supply with oversulfated chondroitin sulfate resulted in several patient deaths (see, e.g., Sasisekharan et al. Thromb Haemost 102: 854-858 (2009)). As a result, a need has arisen for an alternative approach to produce heparin from a defined and controlled source. Current approaches being tested to replace isolation of heparin from slaughter animals include chemical synthesis, chemoenzyme synthesis, and ex vivo cell-based synthesis. However, these approaches have drawbacks as they are costly, technically challenging, and/or do not recapitulate the variety of heparins seen in vivo (see e.g. Laremore et al. Curr Opin Chem Biol 13(5-6): 633-640 (2009)). Development of alternative approaches for producing heparin are needed.

Accordingly, the invention provides a method of producing heparin comprising providing a transgenic non human mammal that has been modified to express heparin in its milk. In one aspect the invention provides a method of producing heparin, the method comprising providing a transgenic non human mammal that has been modified to express one or more heparin biosynthesis enzymes in the mammary gland, and harvesting heparin from the milk produced by the mammary gland of the transgenic mammal

Another aspect of the invention provides a method of producing heparin, the method comprising providing a transgenic non human mammal that has been modified to express one or more heparin biosynthesis enzymes and a core protein in the mammary gland, and harvesting heparin from the milk produced by the mammary gland of the transgenic mammal.

In one aspect, producing heparin in a transgenic mammal refers to increasing expression of heparin above levels already present in the mammal (e.g., producing elevated levels of goat heparin in a transgenic goat). In another aspect, producing heparin in a transgenic mammal refers to producing an ortholog of heparin that does not naturally occur in that mammal (e.g., human heparin produced in a transgenic goat or cow). In another aspect, producing heparin in a transgenic mammal refers to producing a hybrid heparin that does not naturally occur in that mammal (e.g., heparin produced using a combination of goat and human enzymes in a transgenic goat). As heparin and heparin-like molecules have been shown to elicit similar biological responses (e.g., animal heparin used for treatment of human diseases and anti-coagulant properties of invertebrate heparin-like molecules), it should be appreciated that the heparin obtained from a transgenic mammal is not limited to human or non-human mammalian heparin and encompasses heparin variants and heparin-like molecules.

In one aspect, the invention provides a method of producing heparin, the method comprising providing mammary epithelial cells that have been modified to express one or more heparin biosynthesis enzymes, and harvesting heparin from the mammary epithelial cells.

In another aspect, the invention provides a method of producing heparin, the method comprising providing mammary epithelial cells that have been modified to express one or more heparin biosynthesis enzymes and a core protein, and harvesting heparin from the mammary epithelial cells.

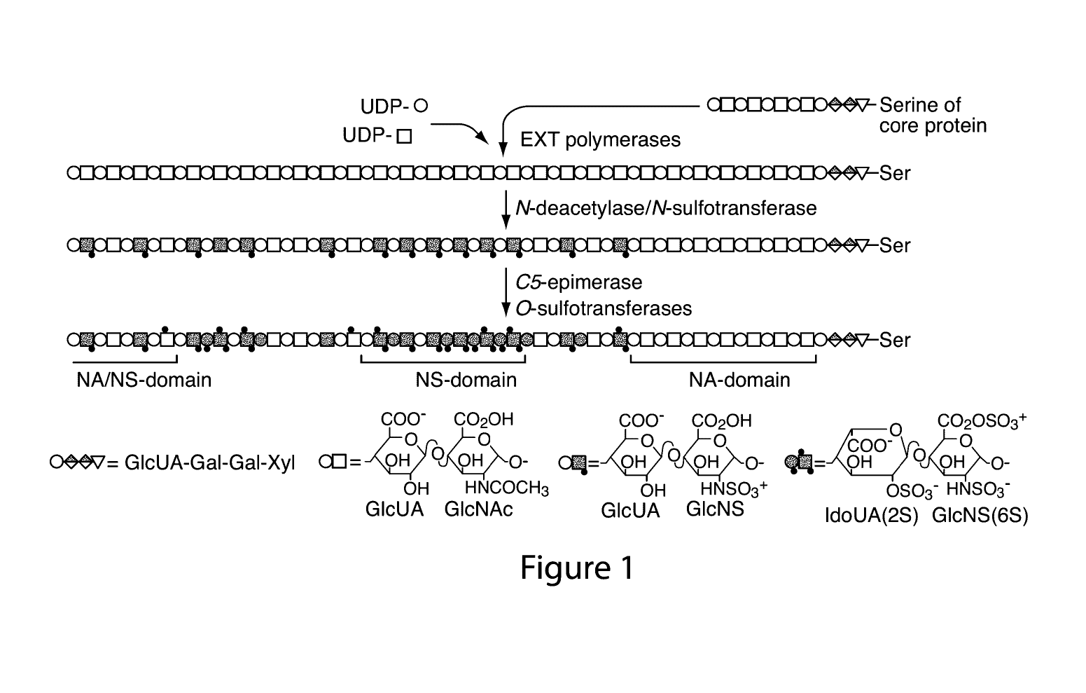

The major enzymes involved in heparin biosynthesis (the “heparin biosynthesis enzymes”) are known in the art (see e.g., Baik et al. Metabolic engineering 14: 81-90 (2012)). The biosynthesis pathway is thought to involve at least twelve enzymatic steps and is outlined in FIG. 1. There is a core protein that is used as the anchor onto which the sugar synthesis takes place. This core protein is transported to the Golgi, where the glycoenzymes reside. Initially there is addition of a tetrasaccharide linkage to Ser residues of the core protein. This is accomplished by sequential addition by four different enzymes that sequentially add xylose, then two gal residues followed by a glucuronic acid. The enzymes that generate the tetrasaccharide are XTI and XTII for xylose addition to Ser on the core protein, B4GalT7 (β1,4-galactosyltransferase), and B3GALT6, (β1,3 galactosyltransferase) which add the gal residues and B3GAT3, (GlcA β1,3-glucuronyltransferase), which adds the glucuronic acid. The enzymes that generate tetrasaccharide are referred to herein as the tetrasaccharide producers and include genes listed in Table 1.

The next step is addition of GlcNAC which establishes that heparin or heparan sulfate will be synthesized on the protein. The enzymes EXTI and EXT2 add GlcNAC and GlcA repeating units in an alternating pattern resulting in the polymerization of heparin. The enzymes that polymerize the chain are referred to herein as the “repeating unit producers” and include the genes listed in Table 2.

The repeating units are modified by GlcNAC N-deacetylase and N-sulfotransferase (e.g. NDST I and NDST II). NDSTs deacetylate and sulfate selected GlcNAC residues to produce GlcNS (NDST II sometimes works with a PAPS sulfate donor). The enzymes used in this step are referred to herein as the repeating units modifiers and include genes listed in Table 3.

In a next step, which may occur before, during or after modification by the repeating units modifiers, the action of C5 epimerase (an “epimerizer”) converts a subset of glucuronic acid (GlcA) to iduronic acid (IdoA). Epimerizers include genes listed in Table 4.

Subsequent to this, a series of sulfotransferases act upon and modify the repeating units, adding sulfate to a subset of the repeating units. These enzymes used in this step are referred to as “sulfation enzymes” and include 3-O-sulfotransferase, 6-O-sulfotransferase, and 2-O-sulfotransferase (3-OST, 6-OST and 2-OST, respectively) and related genes are listed in Table 5.

Following these modifications, the main modified and unmodified disaccharides found within heparin include, but are not limited to, GlcA-GlcNAc, GlcA-GlcNS, IdoA-GlcNS, IdoA(2S)-GlcNS, IdoA-GlcNS(6S), and IdoA(2S)-GlcNS(6S).

Examples of appropriate tetrasaccharide producers include, but are not limited to, the genes in Table 1:

TABLE 1

| Gene | Species | GenBank Number (s) |
|---|---|---|
| XTII (also called | Homo sapiens | NM_022167.2 |
| XYLT2) | Mus musculus | NM_145828.3 |
| | Bos taurus (bovine) | NM_001008667.1 |
| | Capra hircus (goat) | |
| | Cricetulus griseus | |
| | (Chinese hamster) | |
| GalT-1 (also called | Homo sapiens | NM_001497.3 |
| B4GalT7) | | AY358578.1 |
| | Mus musculus | NM_022305.3 |
| | Bos taurus (bovine) | NM_177512.2 |
| | Capra hircus (goat) | HQ700335.1 |
| | Cricetulus griseus | |
| | (Chinese hamster) | |
| GlcAT-1 (also called | Homo sapiens | NM_012200.3 |
| (B3GAT3) | Mus musculus | NM_024256.2 |
| | Bos taurus (bovine) | NM_205805.2 |
| | Capra hircus (goat) | JI861818.1 |
| | Cricetulus griseus | NM_001246684.1 |
| | (Chinese hamster) | |

Examples of appropriate repeating unit producers include, but are not limited to, the genes in Table 2.

TABLE 2

| Gene | Species | GenBank Number (s) |
|---|---|---|
| EXT1 | Homo sapiens | NM_000127.2 |
| | Mus musculus | NM_010162.2 |
| | Bos taurus (bovine) | NM_001098095.1 |
| | Capra hircus (goat) | |
| | Cricetulus griseus | NM_001246767.1 |
| | (Chinese hamster) | |
| EXT2 | Homo sapiens | NM_000401.3 |
| | | NM_001178083.1 |
| | | NM_207122.1 |
| | Mus musculus | NM_010163.3 |
| | Bos taurus (bovine) | NM_177496.3 |
| | Capra hircus (goat) | |
| | Cricetulus griseus | XM_003507774.1 |
| | (Chinese hamster) | |

Examples of appropriate repeating units modifiers include, but are not limited to, the genes in Table 3:

TABLE 3

| Gene | Species | GenBank Number (s) |
|---|---|---|
| NDST1 | Homo sapiens | NM_001543.4 |
| | Mus musculus | NM_008306.4 |
| | Bos taurus (bovine) | NM_001192361.1 |
| | Capra hircus (goat) | |
| | Cricetulus griseus | |
| | (Chinese hamster) | |
| NDST2 | Homo sapiens | NM_003635.3 |
| | Mus musculus | NM_010811.2 |
| | Bos taurus (bovine) | NM_174777.3 |
| | Capra hircus (goat) | |
| | Cricetulus griseus | XM_003496725.1 |
| | (Chinese hamster) | XM_003496726.1 |
| NDST3 | Homo sapiens | NM_004784.2 |
| | Mus musculus | NM_031186.2 |
| | Bos taurus (bovine) | NM_001077961.1 |
| | Capra hircus (goat) | |
| | Cricetulus griseus | XM_003512401.1 |
| | (Chinese hamster) | |
| NDST4 | Homo sapiens | NM_022569.1 |
| | Mus musculus | NM_022565.2 |
| | Bos taurus (bovine) | NM_001192673.1 |
| | Capra hircus (goat) | |
| | Cricetulus griseus | XM_003515214.1 |
| | (Chinese hamster) | |

Examples of appropriate epimerizers include, but are not limited to, the genes in Table 4:

TABLE 4

| Gene | Species | GenBank Number (s) |
|---|---|---|
| C5 epimerase (also | Homo sapiens | NM_015554.1 |
| called GLCE) | Mus musculus | NM_033320.4 |
| | Bos taurus (bovine) | NM_174070.2 |
| | Capra hircus (goat) | |
| | Cricetulus griseus | XM_003498933.1 |
| | (Chinese hamster) | XM_003498932.1 |

Examples of appropriate sulfation enzymes include, but are not limited to, the genes in Table 5:

TABLE 5

| Gene | Species | GenBank Number (s) |
|---|---|---|
| 3-OST-1 (also called | Homo sapiens | NM_005114.2 |
| HS3ST1) | Mus musculus | NM_010474.2 |
| | Bos taurus (bovine) | NM_001076122.1 |
| | Capra hircus (goat) | |
| | Cricetulus griseus | |
| | (Chinese hamster) | |
| 3-OST-2 (also called | Homo sapiens | NM_006043.1 |
| HS3ST2) | Mus musculus | NM_001081327.1 |
| | Bos taurus (bovine) | NM_001205994.1 |
| | Capra hircus (goat) | |
| | Cricetulus griseus | XM_003512052.1 |
| | (Chinese hamster) | |
| 6-OST-1 (also called | Homo sapiens | NM_004807.2 |
| HS6ST1) | Mus musculus | NM_015818.2 |
| | Bos taurus (bovine) | NM_001192491.1 |
| | Capra hircus (goat) | |
| | Cricetulus griseus | XM_003498310.1 |
| | (Chinese hamster) | |
| 6-OST-2 (also called | Homo sapiens | NM_001077188.1 |
| HS6ST2) | Mus musculus | NM_001077202.1 |
| | | NM_015819.3 |
| | Bos taurus (bovine) | NM_001206635.1 |
| | Capra hircus (goat) | |
| | Cricetulus griseus | XM_003506105.1 |
| | (Chinese hamster) | |
| 6-OST-3 (also called | Homo sapiens | NM_153456.3 |
| HS6ST3) | Mus musculus | NM_015820.3 |
| | Bos taurus (bovine) | NM_001205484.1 |
| | Capra hircus (goat) | |
| | Cricetulus griseus | |
| | (Chinese hamster) | |
| 2-OST (also called | Homo sapiens | NM_001134492.1 |
| HS2ST1) | | NM_012262.3 |
| | Mus musculus | NM_011828.3 |
| | Bos taurus (bovine) | XM_002686305.1 |
| | Capra hircus (goat) | |
| | Cricetulus griseus | |
| | (Chinese hamster) | |

Examples of core proteins to be used for synthesis of heparin include, but are not limited, to those listed in Table 6:

TABLE 6

| Gene | Species | GenBank Number (s) |
|---|---|---|
| Serglycin (SRGN) | Homo sapiens | NM_002727.2 |
| | | NR_036430.1 |
| | Mus musculus | NM_011157.2 |
| | Bos taurus (bovine) | NM_001025326.2 |
| | Capra hircus (goat) | |
| | Cricetulus griseus | XM_003499896.1 |
| | (Chinese hamster) | XR_135864.1 |

It should be appreciated that the enzymes may differ in sequence from species to species. Thus, for instance, a bovine NDST I may have a different sequence than a human NDST I. In some embodiments, species specific enzymes are used in the methods described herein. Thus, for instance, goat heparin biosynthesis enzymes are used in transgenic goats. However, in some embodiments, heparin biosynthesis enzymes of one species may be used in a different species. Thus, for instance, human heparin biosynthesis enzymes are used (i.e., expressed) in a transgenic mammal (e.g., transgenic goats). It should further be appreciated that enzymes from different species may be “mixed and matched”. Thus, in some embodiments, a transgenic mammal, such as a transgenic goat, may have both human heparin biosynthesis enzymes and other mammalian heparin biosynthesis enzymes as transgenes.

Heparin Purification from Transgenic Animals

In some embodiments, heparin is purified from the milk of transgenic animals producing heparin. In some embodiments, heparin is purified from the milk of transgenic animals such that the heparin is substantially pure. In some embodiments, substantially pure includes substantially free of contaminants. In some embodiments, contaminants include oversulfated chondroitin sulfate.

In some embodiments, heparin is purified using column chromatography. Column chromatography is well known in the art (see Current Protocols in Essential Laboratory Techniques Unit 6.2 (2008) for general chromotography and U.S. Pat. No. 4,119,774 of purification of heparin). In some embodiments, heparin is purified by immunoprecipitation (see Current Protocols in Cell Biology Unit 7.2 (2001)). In some embodiments, heparin is purified with a heparin-binding antibody or fragment thereof.

Constructs for the Generation of Transgenic Animals Expressing Heparin into Milk

In some embodiments, to produce primary cell lines containing a construct (e.g., encoding one or more heparin biosynthesis enzymes or encoding one or more heparin biosynthesis enzymes and a core protein) for use in producing transgenic animals (e.g. goats) by nuclear transfer, the constructs can be transfected into primary animal skin epithelial cells, for example goat skin epithelial cells, which are clonally expanded and fully characterized to assess transgene copy number, transgene structural integrity and chromosomal integration site. As used herein, “nuclear transfer” refers to a method of cloning wherein the nucleus from a donor cell is transplanted into an enucleated oocyte.

Coding sequences for proteins of interest (e.g., heparin biosynthesis enzymes or heparin biosynthesis enzymes and core protein) can be obtained by screening libraries of genomic material or reverse-translated messenger RNA derived from the animal of choice (such as an ungulate) obtained from sequence databases such as NCBI, Genbank, or by obtaining the sequences of heparin biosynthesis enzymes, etc. The sequences can be cloned into an appropriate plasmid vector and amplified in a suitable host organism, like E. coli.

After amplification of the vector, the DNA encoding the gene can be excised, purified from the remains of the vector and introduced into expression vectors that can be used to produce transgenic animals. After amplification of the vector, the DNA construct can also be excised with the appropriate 5′ and 3′ control sequences, purified away from the remains of the vector and used to produce transgenic animals that have integrated into their genome the desired expression constructs. Conversely, with some vectors, such as yeast artificial chromosomes (YACs), it is not necessary to remove the assembled construct from the vector; in such cases the amplified vector may be used directly to make transgenic animals. The coding sequence can be operatively linked to a control sequence, which enables the coding sequence to be expressed in the mammary gland of a transgenic non-human mammal.

A DNA sequence which is suitable for directing production of heparin biosynthesis enzymes to the milk of transgenic animals can carry a 5′-promoter region derived from a naturally-derived milk protein. This promoter is consequently under the control of hormonal and tissue-specific factors and is most active in lactating mammary tissue. In some embodiments, the promoter is a caprine beta casein promoter. The promoter can be operably linked to a DNA sequence directing the production of a protein leader sequence, which directs the secretion of the transgenic protein across the mammary epithelium into the milk. In some embodiments, a 3′-sequence, which can be derived from a naturally secreted milk protein, can be added to improve stability of mRNA.

As used herein, a “leader sequence” or “signal sequence” is a nucleic acid sequence that encodes a protein secretory signal, and, when operably linked to a downstream nucleic acid molecule encoding a transgenic protein directs secretion. The leader sequence may be the native leader sequence, an artificially-derived leader, or may obtained from the same gene as the promoter used to direct transcription of the transgene coding sequence, or from another protein that is normally secreted from a cell, such as a mammalian mammary epithelial cell.

In some embodiments, the promoters are milk-specific promoters. As used herein, a “milk-specific promoter” is a promoter that naturally directs expression of a gene in a cell that secretes a protein into milk (e.g., a mammary epithelial cell) and includes, for example, the casein promoters, e.g., α-casein promoter (e.g., alpha S-1 casein promoter and alpha S2-casein promoter), β-casein promoter (e.g., the goat beta casein gene promoter (DiTullio et al. BIOTECHNOLOGY 10:74-77 (1992)), 7-casein promoter, κ-casein promoter, whey acidic protein (WAP) promoter (Gordon et al. BIOTECHNOLOGY 5: 1183-1187 (1987)), β-lactoglobulin promoter (Clark et al. BIOTECHNOLOGY 7: 487-492 (1989)) and α-lactalbumin promoter (Soulier et al. FEBS LETTS. 297:13 (1992)). Also included in this definition are promoters that are specifically activated in mammary tissue, such as, for example, the long terminal repeat (LTR) promoter of the mouse mammary tumor virus (MMTV).

As used herein, a coding sequence and regulatory sequences are said to be “operably joined” when they are covalently linked in such a way as to place the expression or transcription of the coding sequence under the influence or control of the regulatory sequences. In order for the coding sequences to be translated into a functional protein the coding sequences are operably joined to regulatory sequences. Two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region were capable of effecting transcription of that DNA sequence such that the resulting transcript might be translated into the desired protein or polypeptide.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids and phagemids. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium, or just a single time per host as the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells, which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase or alkaline phosphatase), and genes which visibly affect the phenotype of transformed or transfected cells, hosts, colonies or plaques. Preferred vectors are those capable of autonomous replication and expression of the structural gene products present in the DNA segments to which they are operably joined.

### Trans Genic Animals and Mammary Epithelial Cells

In one aspect, the disclosure provides a transgenic non human mammal producing heparin in its milk. In particular, the disclosure provides a transgenic non human mammal that expresses one or more heparin biosynthesis enzymes in the mammary gland. A transgenic non human mammal that expresses one or more heparin biosynthesis enzymes and a core protein in the mammary gland is also provided.

In another aspect, mammary epithelial cells that express one or more heparin biosynthesis enzymes are provided. Mammary epithelial cells that express one or more heparin biosynthesis enzymes and a core protein are also provided.

Transgenic animals can be generated according to methods known in the art. In one embodiment, the animals are generated by co-transfecting primary cells with separate constructs. These cells can then be used for nuclear transfer. Alternatively, micro-injection can be used to generate the transgenic animals, and the constructs may be injected.

Animals suitable for transgenic expression include, but are not limited to, goat, sheep, bison, camel, cow, rabbit, buffalo, horse and llama. Suitable animals also include bovine, caprine, and ovine, which relate to various species of cows, goats, and sheep, respectively. Suitable animals also include ungulates. As used herein, “ungulate” is of or relating to a hoofed typically herbivorous quadruped mammal, including, without limitation, sheep, goats, cattle and horses.

Cloning will result in a multiplicity of transgenic animals—each capable of producing the heparin biosynthesis enzymes or other gene construct of interest. The production methods include the use of the cloned animals and the offspring of those animals. In some embodiments, the cloned animals are caprines or bovines. Cloning also encompasses the nuclear transfer of fetuses, nuclear transfer, tissue and organ transplantation and the creation of chimeric offspring.

One step of the cloning process comprises transferring the genome of a cell that contains the transgene encoding one or more heparin biosynthesis enzymes, or one or more biosynthesis heparin enzymes and core protein, into an enucleated oocyte. As used herein, “transgene” refers to any piece of a nucleic acid molecule that is inserted by artifice into a cell, or an ancestor thereof, and becomes part of the genome of an animal which develops from that cell. Such a transgene may include a gene which is partly or entirely exogenous (i.e., foreign) to the transgenic animal, or may represent a gene having identity to an endogenous gene of the animal.

Suitable mammalian sources for oocytes include goats, sheep, cows, rabbits, non-human primates, etc. Preferably, oocytes are obtained from ungulates, and most preferably goats or cows. Methods for isolation of oocytes are well known in the art. Essentially, the process comprises isolating oocytes from the ovaries or reproductive tract of a mammal, e.g., a goat. A readily available source of ungulate oocytes is from hormonally-induced female animals. For the successful use of techniques such as genetic engineering, nuclear transfer and cloning, oocytes may preferably be matured in vivo before these cells may be used as recipient cells for nuclear transfer, and before they were fertilized by the sperm cell to develop into an embryo. Metaphase II stage oocytes, which have been matured in vivo, have been successfully used in nuclear transfer techniques. Essentially, mature metaphase II oocytes are collected surgically from either non-super ovulated or super ovulated animals several hours past the onset of estrus or past the injection of human chorionic gonadotropin (hCG) or similar hormone.

One of the tools used to predict the quantity and quality of the recombinant molecule expressed in the mammary gland is through the induction of lactation (Cammuso, Gavin et al., Animal Biotechnology 11(1): 1-17 (1999)). Induced lactation allows for the expression and analysis of protein from the early stage of transgenic production rather than from the first natural lactation resulting from pregnancy, which could be a year later. Induction of lactation can be done either hormonally or manually.

In some embodiments, the compositions of heparin produced according to the methods provided herein further comprise milk or partially purified milk. In some embodiments, the methods provides herein includes a step of isolating the heparin from the milk of a transgenic animal (See e.g., Pollock et al., Journal of Immunological Methods, 231(1-2): 147-157 (1999)).

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### Examples

### 1. Safety Studies

1.1 Testing the Effect of Heparin on the Mammary Gland

The effect of heparin on the lactating mammary gland was assessed. The level of goat antithrombin (AT) in the mammary gland is about 2 μg/ml, which is 1% of that found in the blood stream. Heparin can be infused into the mammary gland of lactating goats at levels that correspond to the production levels of 1 mg/ml, 200 μg/ml and 50 μg/ml. Since the goat mammary gland can hold 1 liter of milk, the heparin can be infused following milking and the milk removed the following day (infusion of 1 g, 200 mg and 50 mg of high molecular weight heparin). The infusion can be carried out daily for one week or until toxicity is observed in the mammary gland or in the blood.

Levels of heparin can be measured in the milk and in the bloodstream of the animals being tested. Earlier studies have shown that some proteins produced in the mammary gland could leak into the circulation to a level 1% of that found in the milk. The volume of milk can also be monitored throughout the test period to determine if heparin affects lactation. The animals can continue to be milked for another week to test for long-term effects on milk volume. Blood samples can be obtained daily and the concentration of heparin and the coagulation properties monitored by testing for aPTT, (activated partial thromboplastin time). aPTT can be tested in goat blood with various levels of heparin.

A control experiment can be done in which goats are dosed by IV injection of heparin. The level of heparin in the blood that gives rise to bleeding issues can also be determined.

1.2 Toxicity Study of Heparin in Lactating Goats

A study was conducted to determine if the production of heparin in the mammary gland would also have a significant effect on the producing animal. The protein produced by the transgene can potentially affect the mammary gland directly. In addition, it is known that there is potential leakage of the protein into the systemic system. To assess the potential effects of systemic and infused heparin on the goat, the study outlined below was performed. Blood was drawn and milk collected from each milking animal at least once daily during two experiments to determine the effects of heparin administration relative to systemic and udder infusion routes. The levels of heparin, and clotting times as indicated by aPTT were calculated from blood serum. Milk volumes were measured and heparin levels in the milk were determined. In addition, the quality of the milk (color, consistency, evidence of clumping, blood, or mastitis) was noted. The general health of each animal was assessed daily, including a rectal temperature. Since heparin is an anticoagulant, animals were also inspected daily for any gross signs of bleeding, such as abnormal bruising or petechiae.

### Methods

### Animals

Six normal, non-transgenic, lactating goats were fed approximately four pounds of specially formulated vegetarian goat feed, per day, and given a combination of timothy and alfalfa hay as well as free access to water and plain and mineralized salt blocks. As these were lactating animals, they were milked daily starting on the day they arrived. The milk volume was measured each day and a rectal temperature recorded Milk volumes and rectal temperatures prior to the first or second experiment can be seen below in Table 7.

### Blood Collection

Animals were manually restrained so that the jugular vein could be identified. A 21-gauge ¾ inch butterfly needle (BD Vacutainer Blood Collection Set Ref 367251, Franklin Lakes, N.J. USA) was inserted into the vessel, and the blood was collected into 2-1.8 ml sodium citrate vacutainer tubes (BD Vacutainer, Buffered Na Citrate, Franklin Lakes, N.J. USA). After collection (or after blood collection and heparin administration—see below) the needle was removed, and pressure was applied at the collection site to stop any bleeding. The animal received positive reinforcement (grain reward) after each procedure.

### Intravenous Heparin Administration

The animals were restrained as above. The butterfly needle used for blood collection described above is designed so that blood can be collected via a vacutainer blood collection tube, or a syringe can be attached. Thus, after blood collection, the part of the needle that connects with the blood collection tube was removed, and a syringe with heparin was attached and the heparin was slowly injected. An additional 0.4 ml of sterile normal saline was flushed through the needle to ensure that all heparin was delivered. In this way a single needle placement was used for both blood collection and heparin injection, and any additional stress relative to a second venipuncture site was avoided.

TABLE 7
Milk volume in liters and body temperature in degrees Fahrenheit
for all animals prior to the first or second experiment.

| | Animals | | | | | | | | | | | |
|---|---|---|---|---|---|---|---|---|---|---|---|---|
| | Animal 1 | | Animal 2 | | Animal 3 | | Animal 4 | | Animal 5 | | Animal 6 | |
| Day | Milk | Tem | Milk | Tem | Milk | Tem | Milk | Tem | Milk | Tem | Milk | Tem |
| 1 | 0.5 | 102.2 | 0.5 | 101.9 | 3 | 101.9 | 1 | 101.6 | 3 | 102.2 | 3 | 101.9 |
| 2 | 1 | 101.9 | 1.8 | 101.4 | 2.6 | 101.6 | 3.25 | 100.9 | 2.5 | 101.8 | 2.4 | 101 |
| 3 | 2 | 101.2 | 1.75 | 101.2 | 2.75 | 101.2 | 3.5 | 101.2 | 2.75 | 102.1 | 2.25 | 101.4 |
| 4 | 2.5 | 101.2 | 2.25 | 101.9 | 3 | 101.7 | 3.75 | 101 | 2.75 | 101.7 | 2.5 | 101.6 |
| 5 | 2.2 | 102.5 | 2.6 | 102 | 2.4 | 101.9 | 3.5 | 101.2 | 2.5 | 101.8 | 2.3 | 101.4 |
| 6 | 2.1 | 101.8 | 1.9 | 101.3 | 2 | 101.5 | 3.25 | 101 | 2.25 | 101.5 | 2.15 | 102 |
| 7 | 1.75 | 101.6 | 1.6 | 101.5 | 2.05 | 101.7 | 3.15 | 101.3 | 2.1 | 101.7 | 2.5 | 102.1 |
| 8 | 2 | 101.6 | 2.25 | 101.2 | 2.75 | 102 | 3.5 | 101.6 | 2.5 | 101.6 | 2.5 | 102 |
| 9 | 2 | 101.7 | 1.75 | 101.2 | 2.75 | 101.7 | 3 | 101.2 | 2 | 102 | 2.5 | 102.2 |
| 10 | 1.75 | 101.5 | 1.5 | 101.2 | 2 | 102 | 2.75 | 101.4 | 2 | 102.7 | 2.25 | 101.3 |
| 11 | | | | | 2.25 | 101.9 | 3.25 | 101.7 | 2.5 | 101.9 | 2 | 101.4 |
| 12 | | | | | 2.1 | 101.6 | 2.7 | 100.6 | 2.1 | 101.7 | 2.4 | 101.5 |
| 13 | | | | | 2.25 | 101.1 | 2.5 | 101.4 | 2.1 | 101.3 | 2.65 | 101.4 |
| 14 | | | | | 2.25 | 101.7 | 3 | 101.4 | 2.2 | 101.9 | 2.2 | 101.9 |
| 15 | | | | | 2.3 | 101 | 2.75 | 100.4 | 2.15 | 101.1 | 2.4 | 101.8 |
| 16 | | | | | 2.3 | 100.5 | 2.85 | 100.6 | 2.4 | 101.4 | 2.35 | 101 |
| 17 | | | | | 2.6 | 100.9 | 3 | 100.7 | 2.3 | 101.4 | 2.35 | 101.2 |
| Aver | 1.78 | 101.7 | 1.79 | 101.5 | 2.43 | 101.5 | 2.98 | 101.1 | 2.36 | 101.7 | 2.39 | 101.6 |

### Milk Collection

All goats were milked using a portable milking machine, once every day starting the day the goats arrived. Animals were placed on a milking stand for this procedure. The general protocol for milking was as follows. Personnel doing the milking washed their hands thoroughly and donned latex examination gloves. The udder was cleaned using paper towels and water. The udder was then dried using clean paper towels. The teat was cleaned again using alcohol wipes, and pre-dip (FS-106 Sanitizing Teat Dip, IBA Inc., Millbury, Mass. USA) was applied and allowed to dry. A small sample was expressed from the teat by hand to assess milk quality, perform a streak test, and to be frozen for further analysis. The milking machine was then attached to the udder. After milking, the teats were cleaned with alcohol, and post milking disinfectant was applied (Ultra Shield Sanitizing Barrier Teat Dip, IBA Inc., Millbury, Mass. USA). The volume of milk was determined, and the small sample was frozen and stored in a −80° C. chest freezer for subsequent testing.

### Experiment 1: Investigation of the Effects of Systemic Administration of Heparin

Two goats received intravenous (IV) injections of heparin three times daily at 8 AM, 12 noon, and 4 PM for 6 days. The schedule was as follows: low dose of heparin, 1,500 Units (0.15 ml [10,000 units/ml] IV, TID [three times per day]) for two days, followed by a medium dose, 6,000 Units (0.6 ml [10,000 units/ml] IV, TID) for 2 days, ending with a high dose of heparin 31,000 Units (3.1 ml [10,000 units/ml] IV, TID) for 2 days. These doses equate to 1% of the expression level being targeted in the milk/mammary gland of founder transgenic animals to be produced. Blood was taken from the jugular vein prior to each heparin injection. In addition the goats were milked once per day and samples collected. The two goats that received systemic heparin were milked for one day after this experiment before being included in Experiment 2.

### Experiment 2: Udder Infusion of Heparin

The six milking goats were divided into 3 groups of 2 goats. All goats were milked daily with a milking machine, and had blood samples taken. After milking and blood collection, goats had their udders infused with heparin using a teat infusion cannula (Udder Infusion Cannula, IBA Inc., Millbury, Mass. USA). Each group received either a low (25,000 Units), medium (100,000 Units), or high (500,000 Units) dose of infused heparin, equivalent to the 3 different expression levels being explored on a per liter of milk basis. Volumes infused were less than 50 ml per udder. This protocol was performed for a total of seven days. After udder infusions, the goats were milked for an additional eight days, during which blood samples were taken and milk samples collected.

1.3 Results

### Experiment 1

There was a modest rise in aPTTs during the Low and Medium administrations of IV heparin. The aPTTs rose dramatically after administration of the High heparin dose. During the High doses, three of the four noon and 4 PM samples were above the maximum aPTTs that could be calculated by the blood coagulation instrumentation (>124 seconds). The aPTT time in the second High AM sample (prior to AM dose of heparin) had dropped to more normal levels overnight. Milk volumes and body temperatures were not affected by any dose. There was no evidence of petechiae or abnormal bleeding at any time. The only physical finding was an increase in lung sounds in one animal at the noon and 4 PM examinations during the second day of High dose administration. These lung sounds disappeared once the High systemic administrations ended.

### Experiment 2

The results of infused heparin on milk production volume can be analysed as follows. In all cases there is an initial drop in milk volume during the time of heparin infusion into the udder, and appears to be some recovery after cessation of heparin infusions. The largest drop occurred in one animal that received the largest amount of infused heparin (the amount of heparin infused was calculated based on the volume of milk produced prior to infusion) (as indicated in Table 8 below). Table 8 also illustrates the relationship of the amount of heparin infused to the aPTT. aPTT was relative to the amount of heparin infused and appeared to peak during the first 3 days of administration, then diminished. The aPTTs returned to more normal levels (20-40 seconds) during the “washout” period after heparin udder infusion. There did not appear to be a direct correlation of the heparin infusion levels with the post-heparin aPTT levels.

TABLE 8
Milk volumes, units of heparin, and mL infused per udder per day for the
three infusion levels relative to aPTT time. Asterisk (*) indicates 10,000
units/mL, otherwise, 20,000 units/mL. Milk volume is in liters. ML indicates
the mL of heparin infused per udder. aPTT time is in seconds.
Low Dose Udder Infusion (25,000 units/liter)

| | Animal 1 | | | | Animal 6 | | | |
|---|---|---|---|---|---|---|---|---|
| Day | Milk | Units | mL | aPTT | Milk | Units | mlL | aPTT |
| 1 | 2.25 | 56,250 | 2.8* | 23.2 | 2.75 | 68,750 | 3.4* | 26.2 |
| 2 | 2.25 | 56,250 | 2.8* | 36 | 2.25 | 56,250 | 2.8* | 35.5 |
| 3 | 2.2 | 56,250 | 2.8* | 37 | 2.0 | 50,000 | 2.5* | 36.5 |
| 4 | 2.0 | 50,000 | 2.5* | 34.9 | 1.75 | 43,750 | 2.2* | 47 |
| 5 | 1.9 | 50,000 | 2.5* | 34.5 | 1.25 | 31,250 | 1.6* | 55.3 |
| 6 | 1.8 | 43,750 | 2.2* | 34.6 | 1.3 | 31,250 | 1.6* | 41.3 |
| 7 | 1.7 | 43,750 | 2.2* | 33.5 | 1.9 | 50,000 | 2.5 | 32.8 |
| 8 | 2.0 | | | 33.1 | 2.25 | | | 35.9 |

Medium Dose Udder Infusion (100,000 units per liter)

| | Animal 2 | | | | Animal 4 | | | |
|---|---|---|---|---|---|---|---|---|
| Day | Milk | Units | mL | aPTT | Milk | Units | mL | aPTT |
| 1 | 2 | 200,000 | 5 | 30.8 | 3.25 | 300,000 | 8.1 | 24.7 |
| 2 | 1.35 | 135,000 | 7* | 30 | 2.75 | 275,000 | 13.75 | 32.7 |
| 3 | 1.5 | 150,000 | 7.5* | 27.7 | 2.5 | 250,000 | 12.5* | 44 |
| 4 | 1.6 | 150,000 | 7.5* | 31.4 | 1.9 | 200,000 | 10* | 30.2 |
| 5 | 1.3 | 125,000 | 6.25* | 15.4 | 2.2 | 225,000 | 11.25* | 42.4 |
| 6 | 1.2 | 125,000 | 6.25* | 28.3 | 2.0 | 200,000 | 10* | 42.1 |
| 7 | 1.25 | 125,000 | 6.25* | 26.4 | 2.25 | 225,000 | 11.25* | 22.9 |
| 8 | 1.75 | | | 26.9 | 2.5 | | | 29.8 |

High Dose Udder Infusion (500,000 units per liter)

| | Animal 5 | | | | Animal 3 | | | |
|---|---|---|---|---|---|---|---|---|
| Day | Milk | Units | mL | aPTT | Milk | Units | mL | aPTT |
| 1 | 2.25 | 1,125,000 | 28.1 | 23.9 | 3.25 | 1,625,000 | 40.62 | 26.4 |
| 2 | 2.15 | 1,062,500 | 26.5 | 46.9 | 3.0 | 1,500,000 | 37.5 | 41.1 |
| 3 | 1.3 | 625,000 | 16 | >124 | 3.0 | 1,500,000 | 37.5 | >124 |
| 4 | 0.9 | 500,000 | 12.5 | 56 | 1.1 | 0 | 0 | >124 |
| 5 | 1.25 | 625,000 | 15.6 | 50.3 | 0.2 | 0 | 0 | 59.5 |
| 6 | 1.5 | 750,000 | 18.75 | 42 | 0.75 | 0 | 0 | 46.2 |
| 7 | 1.3 | 625,000 | 15.6 | 32.6 | 1.1 | 0 | 0 | 21.3 |
| 8 | 1.9 | | | 34.2 | 1.0 | | | 37.6 |

1.4 Conclusion

### Experiment 1

Systemic administration of heparin is demonstrated herein to have little, if any, effect on body temperature or milk production volume. Low and medium levels of systemic heparin appear to gradually increase aPTTs to 40-50 seconds (normal ranges 20-40). High levels of systemic heparin have a large effect on aPTTs after injection, often rising to coagulation times beyond what could be measured by the machine (>124 sec). But, the aPTTs dropped over night (between the two high doses) to the low 40's, similar to what was seen with the low and medium levels of systemic heparin. There were no obvious gross signs of bleeding, such as petechiae in the oral mucosa, the vulva or udder. In one of the goats during high levels of heparin administration, increased, “crackling” lung sounds could be heard. However, there was no evidence of any blood-tinged exudate from the nose during this time.

From these results, it can be concluded that, surprisingly, systemic heparin administered over 6 days does not cause the petechiae, purpura, or ecchymosis that have been reported as side effects of heparin and doesn't preclude from producing heparin from transgenic mammals.

### Experiment 2

Single daily infusions of low or medium dose heparin into the udder for a week were found to have little effect on body temperature, did not produce any signs of bleeding, and did not increase aPTTs beyond 55 seconds. High levels of infused heparin dramatically increased aPTTs, and infusion of the highest levels appeared to have systemic affects including high body temperature and increased respiratory sounds. The udder of the goat infused with the highest levels appeared to have an inflammatory response, and a dramatic decrease in milk production. However, in this experiment, the goats were milked out and pure heparin was infused, undiluted, directly into the udder. In a transgenic goat producing heparin, the protein is produced with the milk, and is therefore diluted. In addition, heparin produced by the goat itself may not be identical to the exogenous heparin that was infused.

From these studies, it can be concluded that low and medium doses of systemic and infused heparin have minimal effects on goat health and milk production and doesn't preclude from producing heparin from transgenic mammals.

### 2. Generation of Heparin Producing Transgenic Animals

Constructs carrying various modifying enzymes can be introduced into the genome of lines from animals that already produce the core protein. The constructs can be introduced in the order that they are presumed to occur in the pathway, starting with the tetrasaccharide synthesis and ending with the sulfotransferases. At each point (e.g., after the addition of a new enzyme), the heparin can be isolated from the milk of transgenic females.

2.1 Tetrasaccharide Synthesis, EXT I and EXT II, NDST I and NDST II, Sulfotransferases

The DNA coding the enzymes for the enzymes involved in tetrasaccharide synthesis, EXT I and EXT II, NDST I and NDST II, and sulfotransferases can be ligated into the beta casein vector and the constructs microinjected into animal embryos that already carry a core protein. The progeny carrying the new construct can be grown to maturity and tested for the ability to add the tetrasaccharide to the core protein. The genes encoding EXTI and EXTII can also be ligated into the casein vector, and the constructs microinjected into animal embryos that already carry the core protein. Similarly, this approach can be undertaken with the genes encoding, NDST I and NDST II, and the sulfotransferases.

2.2 the Generation of Transgenic Animals with Multiple Heparin Synthesis Pathway Genes

Transgenic lines carrying one group of enzymes (e.g., NDSTI, NDSTII, and C5 epimerase) can be crossed to a separate line of transgenic animal carrying a second group (e.g., core protein and tetrasaccharide synthesis and EXT I and EXT II). The groups of enzymes that are needed to complement the existing heparin synthesis activity of the mammary gland can then be identified from a breeding program.

In a separate, parallel program, a large construct (using BAC or YAC) can be assembled that carries all of the genes required for the pathway. This large construct can then be introduced into the animal genome. This method has been used successfully to construct transgenic animals carrying large multi-gene arrays (e.g., immunoglobulin loci).

2.3 Secretion Process

Most of the glycosaminoglycan proteins are membrane bound or intracellular.

A soluble form can also be engineered to aid in secretion. For example, some GAG proteins (glypican) are linked to cell surface with a GPI anchor. The anchor site can be deleted so that the soluble version can be produced.

Fat globule secretion: Membrane proteins can be produced in fat globule membranes.

2.4 Generation of Large Transgenic Animals that Produce Heparin

The constructs can be introduced into either the goat or cow genome by somatic cell nuclear transfer (cloning). Transgenic goats have the advantage of shorter generation times compared to cows. However transgenic cows generate roughly 10-fold more milk per animal and provide enhanced scalability. Furthermore, although cows have longer generation time than goats, significant amounts of milk can be obtained through hormonal induction of juvenile calves, erasing some of the impact of generation time on development timelines.

Large expression constructs can be transfected into cells, such as fibroblasts, and selection for successful clones can be done. These cloned cells can be then screened for the successful incorporation of all of the relevant heparin biosynthesis genes. The cloning procedure can result in a number of founders carrying the heparin pathway. Induction at 2 months for the goat (or 6 months for bovine) can be performed to determine if core protein was successfully modified to form heparin in the mammary gland of the large animal.

### EQUIVALENTS

The foregoing written specification is considered to be sufficient to enable one skilled in the art to practice the invention. The present invention is not to be limited in scope by examples provided, since the examples are intended as an illustration of certain aspects and embodiments of the invention. Other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. A method of producing heparin, the method comprising
providing a non human transgenic mammal that produces heparin in its milk, and
harvesting heparin from the milk produced by the mammary gland of the transgenic mammal.

2. A method of producing heparin, the method comprising
providing a non human transgenic mammal that has been modified to express one or more heparin biosynthesis enzymes in the mammary gland, and
harvesting heparin from the milk produced by the mammary gland of the transgenic mammal.

3. A method of producing heparin, the method comprising
providing a non human transgenic mammal that has been modified to express one or more heparin biosynthesis enzymes and core protein in the mammary gland, and
harvesting heparin from the milk produced by the mammary gland of the transgenic mammal.

4. The method of any one of claims 1-3, wherein the transgenic mammal is an ungulate.

5. The method of claim 4, wherein the ungulate is a goat.

6. The method of claim 5, wherein the ungulate is a bovine.

7. A method of producing heparin, the method comprising
providing mammary epithelial cells that have been modified to express one or more heparin biosynthesis enzymes, and
harvesting heparin from the mammary epithelial cells.

8. A method of producing heparin, the method comprising
providing mammary epithelial cells that have been modified to express one or more heparin biosynthesis enzymes and core protein, and
harvesting heparin from the mammary epithelial cells.

9. A transgenic non human mammal that produces heparin in its milk.

10. A transgenic non human mammal that has been modified to express one or more heparin biosynthesis enzymes in its mammary gland.

11. A transgenic non human mammal that has been modified to express one or more heparin biosynthesis enzymes and core protein in its mammary gland.

12. A transgenic non human mammal as defined in any one of the preceding claims.

13. Mammary epithelial cells that have been modified to express one or more heparin biosynthesis enzymes.

14. Mammary epithelial cells that have been modified to express one or more heparin biosynthesis enzymes and core protein.

15. The method, mammal or cells of any one of the preceding claims, wherein one or more of the heparin biosynthesis enzymes are under the control of a milk promoter.

16. The method, mammal or cells of claim 15, wherein the milk promoter is a goat beta casein promoter.

17. Heparin produced according to any of the methods of the preceding claims.

18. A composition comprising the heparin of claim 17.

19. The composition of claim 18, further comprising milk.
